SEQUENCE LISTING (6 sequences)

SEQ ID NO: 1 - DNA, 40 bp, Artificial Sequence
  Description of Artificial Sequence Synthetic XO1 oligonucleotide
acgtcgaccgatgaatagcggtcagatccgtacctactcg

SEQ ID NO: 2 - DNA, 40 bp, Artificial Sequence
  Description of Artificial Sequence Synthetic XO2 oligonucleotide
acgtcgagtaggtacggatctgcgtattgcgaacgactcc

SEQ ID NO: 3 - DNA, 40 bp, Artificial Sequence
  Description of Artificial Sequence Synthetic XO3 oligonucleotide
acgtcgagtcgttcgcaatacggctgtacgtatggtctcc

SEQ ID NO: 4 - DNA, 40 bp, Artificial Sequence
  Description of Artificial Sequence Synthetic XO4 oligonucleotide
acgtcgagaccatacgtacagcaccgctattcatcggtcg

SEQ ID NO: 5 - DNA, 32 bp, Artificial Sequence
  Description of Artificial Sequence Synthetic oligonucleotide
tcgaaccatggtgagcaagggcgccgagctgt

SEQ ID NO: 6 - DNA, 31 bp, Artificial Sequence
  Description of Artificial Sequence Synthetic oligonucleotide
tgtctggtgtcaaaatagggggggggggttc

(19) United States
(12) Patent Application Publication
(10) Pub. No.: US 2015/0050698 A1
(43) Pub. Date: Feb. 19, 2015

(54) DEVELOPMENT OF NUCLEIC ACID GEL MATRIX FOR CELL-FREE PROTEIN SYNTHESIS OF CELL NUCLEUS REPLICATE, AND METHOD FOR PRODUCING SAME

(71) Applicant: RESEARCH & BUSINESS FOUNDATION SUNGKYUNKWAN UNIVERSITY, Suwon-si (KR)

(72) Inventors: Soong Ho Um, Seoul (KR); Sun Ju Bae, Suwon-si (KR)

(73) Assignee: RESEARCH & BUSINESS FOUNDATION SUNGKYUNKWAN UNIVERSITY, Suwon-si (KR)

(21) Appl. No.: 14/359,000

(22) PCT Filed: Sep. 21, 2012
(86) PCT No.: PCT/KR2012/007604

§ 371 Date: May 16, 2014

(30) Foreign Application Priority Data
Nov. 17, 2011 (KR) 10-2011-0120120

(51) Int. Cl. C12N 15/10; C12N 15/63; C12P 21/00

(52) U.S. Cl. CPC C12N 15/10; C12P 21/00; C12N 15/63
USPC 435/91.41; 435/320.1; 435/183

(57) ABSTRACT

Provided are a nucleic acid gel matrix for the cell-free protein synthesis of a cell nucleus replicate that contains an X-type nucleic acid nanostructure, an expression plasmid containing a DNA fragment, transcription and translation constituents, and a lipid membrane component, and a method for producing same. The nucleic acid gel matrix for the cell-free synthesis of a cell nucleus replicate is a polymorphic gel matrix that contains the X-type nucleic acid nanostructure, the expression plasmid containing the DNA fragment, the transcription and translation constituents, and the lipid membrane component.

### CROSS-REFERENCE TO RELATED APPLICATIONS

This application is a National Stage of International Application No. PCT/KR2012/007604 filed Sep. 21, 2012, claiming priority based on Korean Patent Application No. 10-2011-0120120 filed Nov. 17, 2011, the contents of all of which are incorporated herein by reference in their entirety.

### BACKGROUND

1. Field

The following description relates to development of a nucleic acid matrix for synthesizing a nucleus mimetic cell-free protein, including an X-type nucleic acid nano structure, an expression plasmid including a DNA fragment, transcription and translation factors and lipid membrane components, and a method of preparing the same.

2. Description of Related Art

Recently, as an attempt to reproduce excellent functional systems of nature, studies on highly integrated technology-biomimetic engineering (bio-inspired engineering) in which multifunctional studies including biochemistry, physics, engineering. mathematics, etc. are compiled is actively progressing. This technology is rapidly propagating into various fields including medical and bio-related engineering, fiber engineering, and food engineering. For example, a study for increasing low transferring efficiency which has been a problem in endocytosis shows a more improved result by mimicking an excellent transferring ability of a natural virus.

Likewise, every material present in nature attracts attention as an object of study of biomimetic engineering. Particularly, the smallest constituent unit of life, the cell, is a metabolic result, and an important subject for controlling a biometric system, and thus efforts to study and replicate functions of cells are increasing every day. Representative cell mimetic studies that have progressed so far include cell wall lipid membrane mimetic studies, studies on mimicking a nuclear pore carrier for selective exchange between multispecies materials or cell wall lipid membranes, and studies on artificial dendritic cells for controlling an immune signal system. In line with the trend of the above studies, an object of the present invention is to mimic a function of a chromosome, which is a subject of producing a protein in a nucleus and maximizing conventional commercial protein production capacity.

Currently known commercial protein production methods are mainly methods of disrupting a cytoplasm of a living cell or chemically polymerizing an amino acid. However, such a method has disadvantages of a very complicated production process in cell lysis and purification/isolation, abnormal expression of the prepared protein, an immune reaction since the protein is not taken well in the living system, and decreased accuracy of an amino acid sequence in polymerization of a long-chain protein. Accordingly, development of a cell-free protein synthesis system (or in vitro protein synthesis system) not using cells has been actively progressing recently.

The cell-free protein synthesis system is an in vitro method for synthesizing a protein using critical factors (adenosine triphosphate (ATP), an amino acid, etc) necessary for synthesis of a protein by adding a substrate or an enzyme to a cell lysate or extract. Such a cell-free protein synthesis system is expected to enable synthesis of any protein since it can overcome disadvantages of a conventional method of producing a protein using cells. In addition, such a system is also expected to increase productivity since it is free from various external conditions such as pH, temperature, and ion strength when a protein is synthesized. However, although the system has such advantages, the system is not substantially used due to a high production cost of a reagent consumed due to short reaction time and a low protein production rate. To overcome such a disadvantage, various attempts to add various chemicals such as iodoacetamide, glutathione, etc., or to increase productivity by developing a continuous moving system have been made, but the system is still not sufficient to be industrially used.

There is a demand for developing an effective cell-free protein synthesis system that can stably maintain high productivity.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a nucleic acid gel matrix for synthesizing a nucleus mimetic cell-free protein includes an X-type nucleic acid nano structure, an expression plasmid including a DNA fragment, transcription and translation factors, and lipid membrane components.

The X-type nucleic acid nano structure may be formed by hybridizing DNA sequences of SEQ. ID. NOs: 1, 2, 3, and 4.

The DNA fragment may be an Aequorea coerulescens green fluorescent protein (AcGFP) gene.

The DNA fragment may be a gene related to cancer cell death or angiogenesis.

The gene may be selected from the group consisting of human sonic hedgehog (hShh), tumor necrosis factor-related apoptosis-inducing ligand (hTRAIL), human glail-derived neurotrophic factor (hGDNF), and mouse-stromal cell-derived factor-1α (SDF1α) genes.

The DNA fragment may be a gene related to production of a mussel adhesive protein (MAP).

The gene may be a fp-151 gene.

The DNA fragment may be a gene related to production of CD44, that is, a cancer stem cell marker.

The expression vector including the DNA fragment may be a plasmid in vitro expression (pIVEX) vector, that is, a wheat germ or E. coli based system.

The transcription and translation factors may include an RNA polymerase (T7 RNA polymerase), an ATP, an NTP mixed solution, a cell lysate, and an amino acid.

The lipid membrane components may be 1,2-dioleoyl-sn-glycero-3-phosphocholine (DOPC), 1,2-dioleoyl-3-trimethylammonium-propane (DOTAP), and 1,2-dihexadecanoyl-sn-glycero-3-phosphoethanolamine (Texas-red DHPE).

The nucleic gel matrix may have any of various shapes in which each size of width, length and height is 100 nm to 1,000 μm, or a maximum diameter is 100 μm or less.

In another general aspect, there is provided a method of preparing a nucleic acid gel matrix for synthesizing a nucleus mimetic cell-free protein, involving the step of: (a) forming a superhydrophobic surface by depositing zinc oxide (ZnO) nanoparticles on a sapphire substrate, depositing gold on the surface through e-beam lithography or vacuum sputtering, and treating 1 mM of a heptadecafluoro-1-decanethiol (HDFT) solution at room temperature for 15 minutes; (b) preparing an X-type nucleic acid nano structure by combining single strand nucleic acids of SEQ. ID. NO: 1, 2, 3, and 4 through complementary hybridization of base sequences; (c) preparing a recombinant plasmid by combining a plasmid in vitro expression (pIVEX) vector with a DNA fragment using restriction enzymes and a T4 DNA ligase; (d) preparing a nucleic acid gel precursor by combining the X-type nucleic acid nano structure, the recombinant plasmid, and the transcription and translation factors using a T4 DNA ligase; (e) plating the nucleic acid gel precursor solution on the superhydrophobic surface, covering the surface with a PDMS template, and performing a reaction at room temperature for at least 4 hours; and (f) reacting the prepared nucleic acid matrix with a liposome prepared with lipid membrane components (DOPC, DOTAP, and Texas-red DHPE) at room temperature for at least 2 hours.

The DNA fragment may be an AcGFP gene.

The DNA fragment may be a gene related to cancer cell death or angiogenesis.

The gene may be selected from the group consisting of human sonic hedgehog (hShh), tumor necrosis factor-related apoptosis-inducing ligand (hTRAIL), human glail-derived neurotrophic factor (hGDNF), and mouse-stromal cell-derived factor-1α (SDF1α) genes.

The DNA fragment may be a gene related to production of a mussel adhesive protein (MAP).

The gene may be a fp-151 gene.

The DNA fragment may be a gene related to production of CD44, that is, a cancer stem cell marker.

The expression vector including the DNA fragment may be a pIVEX vector, that is, a wheat germ or E. coli based system.

The transcription and translation factors may include an RNA polymerase (T7 RNA polymerase), an ATP, an NTP mixed solution, a cell lysate, and an amino acid.

The lipid membrane components may be 1,2-dioleoyl-sn-glycero-3-phosphocholine (DOPC), 1,2-dioleoyl-3-trimethylammonium-propane (DOTAP), and 1,2-dihexadecanoyl-sn-glycero-3-phosphoethanolamine (Texas-red DHPE).

Other features and aspects will be apparent from the following detailed description, the drawings, and the claims.

### BRIEF DESCRIPTION OF THE DRAWINGS

FIG. 1 shows (a) a schematic diagram of an X-type nucleic acid nano structure and (b) a diagram showing electrophoresis results thereof.

FIG. 2 shows (a) and (b) electrophoresis results obtained in preparation of a recombinant plasmid, (c) a diagram showing a sequencing result thereof, and (d) and (e) diagrams showing electrophoresis results obtained in preparation of a recombinant plasmid with respect to genes related to cancer cell death and an angiogenesis.

FIG. 3 is a schematic diagram of a nucleic acid matrix.

FIG. 4 is a diagram showing a result of preparing a nucleic acid gel matrix including components necessary for transcription and translation.

FIG. 5 is a schematic diagram showing a method of preparing a superhydrophobic surface.

FIG. 6 is a diagram showing results of measuring surface hydrophobicity according to an HDFT concentration and a preparing method.

FIG. 7 is a diagram showing a result obtained by observing formation of a hydrophobic surface using a scanning electron microscope.

FIG. 8 is a diagram showing a result obtained by observing a nucleic acid gel matrix for synthesizing a cell-free protein (oval shape) using a fluorescent microscope.

FIG. 9 shows a schematic diagram showing a preparation of a nucleic acid matrix for synthesizing a nucleus mimetic cell-free protein by combination with lipid membrane components, and a diagram showing a result obtained by observing the prepared nucleic acid matrix using a confocal microscope.

FIG. 10 is a diagram showing results obtained by estimating protein expression efficiency of the preparation gel matrix for synthesizing a cell-free protein (oval shape).

FIG. 11 shows diagrams showing (a) and (d) results of comparing protein expression of a conventional solution-phase cell-free protein synthesis system and the nucleic acid gel matrix for synthesizing a cell-free protein according to the present invention, (b) protein expression levels according to a combining ratio of an X-type nucleic acid nano structure and a recombinant plasmid, and (c) a protein expression level according to an incubation time.

FIG. 12 is a diagram identifying molecular weights of proteins produced from the conventional solution-phase cell-free protein synthesis system and the nucleic acid gel matrix for synthesizing a cell-free protein according to the present invention through electrophoresis.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art.

### Technical Problem

The present invention is provided to maximize protein production capacity by basically mimicking physical arrangement of genes in a cell nucleus as well as solving the above-described problems of the conventional arts. Therefore, the present invention is directed to providing development of a nucleic acid gel matrix for synthesizing a nucleus mimetic cell-free protein including an X-type nucleic acid nano structure, an expression plasmid including a DNA fragment, transcription and translation factors and a lipid membrane component, and a method of preparing the same in order to prepare an artificial cell core.

However, the technical object of the present invention to be achieved is not limited to the above-described object, and other objects not described herein will be clearly understood by those of ordinary skill in the art with reference to the following descriptions.

### Technical Solution

One aspect of the present invention provides a nucleic acid gel matrix for synthesizing a nucleus mimetic cell-free protein including an X-type nucleic acid nano structure, an expression plasmid including a DNA fragment, transcription and translation factors, and lipid membrane components.

Another aspect of the present invention provides a method of preparing the nucleic acid gel matrix for synthesizing a nucleus mimetic cell-free protein, which includes preparing a superhydrophobic surface by depositing zinc oxide (ZnO) nanoparticles on a sapphire substrate, depositing gold on the surface through e-beam lithography or vacuum sputtering, and treating 1 mM of a heptadecafluoro-1-decanethiol (HDFT) solution at room temperature for 15 minutes; preparing an X-type nucleic acid nano structure by combining single strand nucleic acids of SEQ. ID. NOs: 1, 2, 3 and 4 through complementary hybridization of base sequences; preparing a recombinant plasmid by combining a plasmid in vitro expression (pIVEX) vector and a DNA fragment with a reaction enzyme and a T4 DNA ligase; preparing a nucleic acid gel precursor by combining the X-type nucleic acid nano structure, the recombinant plasmid, and the transcription and translation factors using a T4 DNA ligase; plating the nucleic acid gel solution on the superhydrophobic surface, covering the surface with a PDMS template, and performing a reaction at room temperature for at least 4 hours; and reacting the prepared nucleic acid matrix with a liposome prepared with the lipid membrane components (DOPC, DOTAP, and Texas-red DHPE) at room temperature for at least 2 hours.

In one embodiment of the present invention, in the X-type nucleic acid nano structure, the DNA sequences of SEQ. ID. NOs: 1, 2, 3, and 4 are hybridized.

In another embodiment of the present invention, the DNA fragment is an Aequorea coerulescens green fluorescent protein (AcGFP) gene.

In still another embodiment of the present invention, the DNA fragment is a gene related to cancer cell death or angiogenesis. Preferably, the gene is selected from the group consisting of human sonic hedgehog (hShh), tumor necrosis factor-related apoptosis-inducing ligand (hTRAIL), human glail-derived neurotrophic factor (hGDNF), and mouse-stromal cell-derived factor-1α (SDF1α) genes.

In yet another embodiment of the present invention, the DNA fragment is a gene related to production of a mussel adhesive protein (MAP), and preferably an fp-151 gene.

In yet another embodiment of the present invention, the DNA fragment is a gene related to production of a cancer stem cell marker, CD44.

In yet another embodiment of the present invention, the expression plasmid including the DNA fragment is a pIVEX vector, which is a wheat germ or E. coli based system.

In yet another embodiment of the present invention, the transcription and translation factors include a T7 RNA polymerase, an ATP and NTP-mixed solution, a cell lysate, and an amino acid.

In yet another embodiment of the present invention, the lipid membrane components include 1,2-dioleoyl-sn-glycero-3-phosphocholine (DOPC), 1,2-dioleoyl-3-trimethylammonium-propane (DOTAP), and 1,2-dihexadecanoyl-sn-glycero-3-phosphoethanolamine (Texas-red DHPE).

In yet another embodiment of the present invention, the nucleic acid gel matrix has any of various shapes in which each size of width, length and height is 100 nm to 1,000 μm, or a maximum diameter is 100 μm or less.

### Advantageous Effects

A nucleic acid gel matrix for synthesizing a nucleus mimetic cell-free protein according to the present invention is a structure of various shapes including an expression plasmid including an X-type nucleic acid nano structure, DNA fragment, transcription and translation factors, and lipid membrane components. Because of a structural characteristic of the nucleic acid gel matrix, DNA, transcription and translation factors, etc. are massed in a small space, which is similar to a natural genome massed structure in a cell, thereby enabling large amounts of proteins to be produced. This structure provides more improvement in protein production capacity than a currently commercialized cell-free protein synthesis method. In addition, since the structure is prepared to protect external genomes corresponding to treatment of various diseases, which are included in a gel, when being injected into a body, it is expected to be original technology for enhancing therapeutic effects such as stable anticancer treatment and treatment of inflammation. In addition, the nucleus mimetic system prepared by the combination of the developed nucleic acid matrix and the lipid membrane components may be suggested as a next generation platform for future gene therapy since it is a naturally structured mimic which has hardly any toxicity or immune responses and therefore can replace an existing nuclear system of a cell.

### Modes of Invention

The inventors studied an effective nucleic acid gel matrix system for synthesizing a nucleus mimetic cell-free protein, which can stably maintain high protein productivity, and thereby completed the present invention.

The present invention provides a nucleic acid gel matrix for synthesizing a nucleus mimetic cell-free protein, which includes an expression plasmid including an X-type nucleic acid nano structure, DNA fragment, transcription and translation factors, and lipid membrane components.

The nucleic acid gel matrix for synthesizing a nucleus mimetic cell-free protein according to the present invention is formed in any of various shapes. In one embodiment of the present invention, to prepare various shapes of gel matrixes, simply, oval, circular, and rectangular PDMS molds were used. Since a shape of gel may be changed depending on a shape of a mold, there is no limit to a transformable shape of the gel. Preferably, the gel has an oval, circular or rectangular shape. More preferably, the surface area may be relatively large and circular or oval. There is no limit to a size of the nucleic acid gel matrix, but preferably the nucleic acid gel matrix is a structure of a shape in which each size of a width, length or height is 100 nm to 1,000 μm, or a maximum diameter is 100 μm or less.

As a method of preparing an oval or circular gel matrix having a relatively large surface area, a method of putting a mold on a superhydrophobic substrate is used. In the present invention, a method of preparing the nucleic acid matrix for synthesizing a nucleus mimetic cell-free protein includes (a) preparing an X-type nucleic acid nano structure by depositing zinc oxide (ZnO) nanoparticles on a sapphire substrate, coating the surface with gold through e-beam lithography or sputtering, and treating 1 mM of a HDFT solution at room temperature for 15 minutes; (b) preparing an X-type nucleic acid nano structure by combining single strand nucleic acids of SEQ. ID. NOs: 1, 2, 3 and 4 by complementary hybridization of base sequences; (c) preparing a recombinant plasmid by combining a pIVEX vector and a DNA fragment using a restriction enzyme and a T4 DNA ligase; (d) preparing a nucleic acid gel precursor by combining the X-type nucleic acid nano structure, the recombinant plasmid and the transcription and translation factors using a T4 DNA ligase; (e) plating the gel precursor solution on the superhydrophobic surface, covering the surface with a mold PDMS template, and performing the reaction at room temperature for at least 4 hours; and (f) reacting the prepared gene network hydrogenated gel with a liposome prepared with the lipid membrane components (DOPC. DOTAP, and Texas-red DHPE) at room temperature for 2 hours.

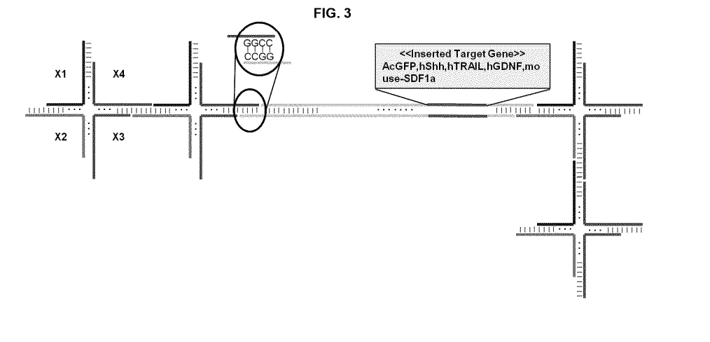

The inventors confirmed that the nucleic acid matrix may be prepared using the X-type nucleic acid nano structure in which DNA sequences of SEQ. ID. NOs: 1, 2, 3 and 4 are combined, and a gene coding for a target protein may be hybridized within the nucleic acid nano structure. Accordingly, there is no limit to a kind of the gene that may be hybridized within the X-type nucleic acid nano structure. Preferably, the gene may be a gene related to cancer cell death or angiogenesis (an hShh, hTRAIL, hGDNF, or SDF1α gene). In addition, the gene may be a gene related to production of a cancer stem cell, i.e., CD44, or a gene related to production of a mussel adhesive protein (MAP, e.g., an fp-151 gene). However, the present invention is not limited. A schematic diagram of the nucleic acid matrix is shown in FIG. 3.

The expression plasmid including the DNA fragment is a pIVEX, that is, a wheat germ or E. coli-based system. Preferably, the wheat germ-based system is a pIVEX 1.3WG or pIVEX 1.4WG series, and the E. coli-based system is pIVEX 2.3E. coli or pIVEX 2.4E. coli.

In addition, the inventors prepared a gel structure of various shapes (e.g., oval, circle or rectangle) including transcription and translation factors to prepare a gel matrix for cell-free protein expression using only the nucleic acid, and used the structure to express a nucleic acid matrix-phase cell-free protein.

In one embodiment of the present invention, it was confirmed that a protein production capacity of the nucleic acid gel matrix for synthesizing a nucleus mimetic cell-free protein is 20 times higher than that of the conventional solution-phase system, the gel matrix is stably recycled, and the protein is expressed for a long time (refer to Example 3).

Consequently, the nucleic acid gel matrix for synthesizing a nucleus mimetic cell-free protein is expected to be applied in gene therapy against various diseases. In addition, since the gel matrix has a micro-unit small size and DNA is included in the structure to protect from an external factor, the matrix is expected to stably express a protein in a human body.

Hereinafter, exemplary examples will be provided to help understanding of the present invention. However, the following examples are merely so that the present invention can be more easily understood, and the scope of the present invention is not limited to the following examples.

### EXAMPLES

### Example 1

### Preparation of Nucleic Acid Gel Matrix

1-1. Preparation of X-Type Nucleic Acid Nano Structure

To prepare a nucleic acid matrix, the preparation of four kinds of single strand nucleic acids was entrusted to Integrated DNA Technology. Sequences of the prepared single strand nucleic acids are as follows:

X01
(5′-ACGTCGACCGATGAATAGCGGTCAGATCCGTACCTACTC
G-3′, SEQ. ID. NO: 1)
X02
(5′-ACGTCGAGTAGGTACGGATCTGCGTATTGCGAACGACTC
C-3′, SEQ. ID. NO: 2)
X03
(5′-ACGTCGAGTCGTTCGCAATACGGCTGTACGTATGGTCTC
C-3′, SEQ. ID. NO: 3)
X04
(5′-ACGTCGAGACCATACGTACAGCACCGCTATTCATCGGTC
G-3′, SEQ. ID. NO: 4)

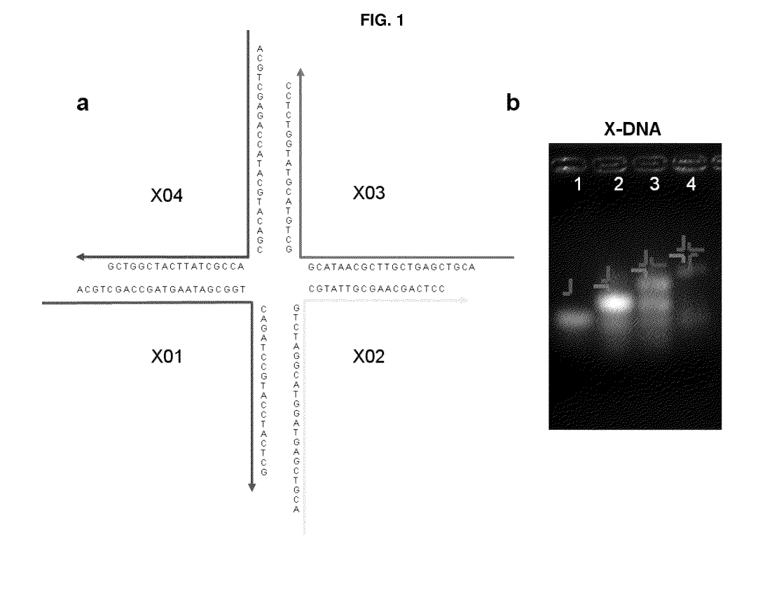

An X-type nucleic acid nano structure was prepared by inducing complementary hybridization of the base sequence (annealing using a thermocycler, reducing temperatures: 2 minutes at 95° C.→2 minutes at 65° C.→5 minutes 30 seconds at 60° C.→reducing temperature by −1° C. every 30 seconds from 60° C.→30 seconds at 20° C.→reducing temperature from 20° C. to 4° C.). A schematic diagram of the X-type nucleic acid nano structure is shown in FIG. 1. In addition, each binding nucleic acid nano structure was identified through electrophoresis. The result is shown in FIG. 1(b).

As shown in FIG. 1(b), it is confirmed that the first lane is an X01 single strand, the second lane is a structure in which X01 and X02 are hybridized, the third lane is a structure in which X01, X02, and X03 are hybridized, and the fourth lane is an X-type nucleic acid nano structure in which X01 to X04 are hybridized. According to the results, it can be confirmed that each single strand nucleic acid becomes larger as they are hybridized, and therefore the X-type nucleic acid nano structure is prepared by hybridizing four kinds of single strand nucleic acids.

1-2. Preparation of Recombinant Plasmid

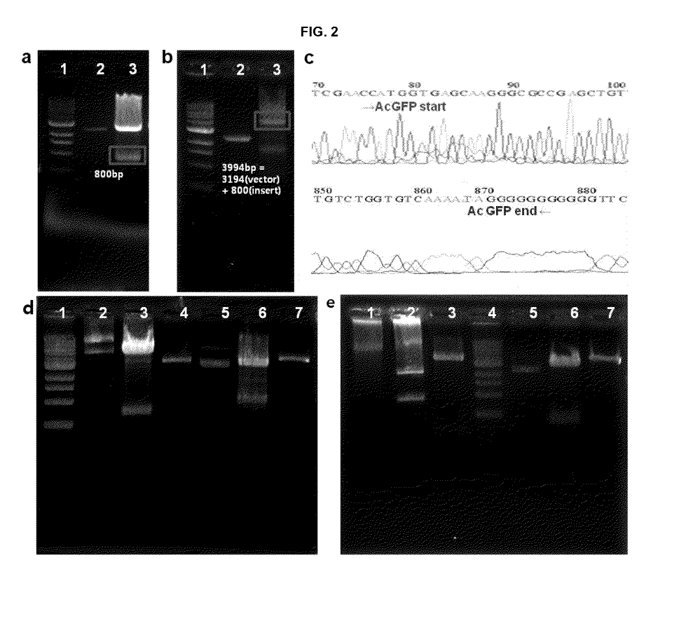

To prepare a recombinant plasmid to be expressed in the nucleic acid gel matrix for synthesizing a nucleus mimetic cell-free protein, each of a plasmid including an AcGFP gene sequence expressing a green fluorescent protein (GFP) used as a conceptual model and a pIVEX vector was digested with restriction enzymes (Nco I and Stu I/Nco I and Sma I for AcGFP plasmid/pIVEX vector), and the AcGFP gene and the pIVEX vector were bound with a T4 DNA ligase at 18 to 22° C. for 16 hours. The results are shown in FIG. 2.

As shown in FIG. 2, it was confirmed that the AcGFP-including plasmid was digested with a restriction enzyme (800 bp) (refer to FIG. 2(a)). In addition, it was confirmed that 3994 by of a band (recombinant plasmid) was formed by hybridizing a digested AcGFP gene with a pIVEX vector (refer to FIG. 2(b)).

As shown in FIG. 2(c), it was confirmed that the recombinant plasmid was prepared by normally inserting the AcGFP gene into the pIVEX vector through DNA sequencing.

In addition to the AcGFP gene, the recombinant plasmid was prepared by the same method using an hShh, hTRAIL, hGDNF, or SDF1α gene, which are related to cancer cell death and angiogenesis (refer to FIGS. 2(d) and (e)).

As shown in FIG. 2(d), it can be confirmed that an hShh gene-inserted plasmid (the second lane) is digested with the restriction enzymes such as Hind III and Not I, thereby generating a linear hShh (the third lane) gene sequence. It can be confirmed that the hTRAIL gene-inserted plasmid (the fifth lane) is digested with restriction enzymes such as Nco I and Hpa I, thereby generating a linear h TRAIL (the sixth lane) gene sequence. In addition, it can be confirmed that the pIVEX vector is digested with corresponding restriction enzymes, as in the fourth and seventh lanes, to enable to be recombined to each gene.

As in FIG. 2(e), it can be confirmed that an hGDNF gene-inserted plasmid (the first lane) was digested with the restriction enzymes such as Hind III and Hpa I, thereby generating a linear hGDNF (the second lane) gene sequence. In addition, it can be confirmed that a mouse-SDF1α gene-inserted plasmid (the fifth lane) was digested with the restriction enzymes such as Not I and Sal I, thereby generating a mouse-SDF1α (the sixth lane) gene sequence. In addition, it can be confirmed that the pIVEX vector is digested with corresponding restriction enzymes, as in the third and seventh lanes, to enable recombination to each gene.

1-3. Preparation of Nucleic Acid Gel Matrix

To prepare a nucleic acid gel matrix, an X-type nucleic acid nano structure of Example 1-1 and a recombinant plasmid of Example 1-2 were hybridized with a T4 DNA ligase at room temperature for at least 4 hours. A schematic diagram of the nucleic acid matrix is shown in FIG. 3.

As shown in FIG. 3, a nucleic acid gel matrix was prepared by forming a matrix based on an X-type nucleic acid nano structure, and binding a recombinant plasmid including a target gene within the X-type nucleic acid nano structure.

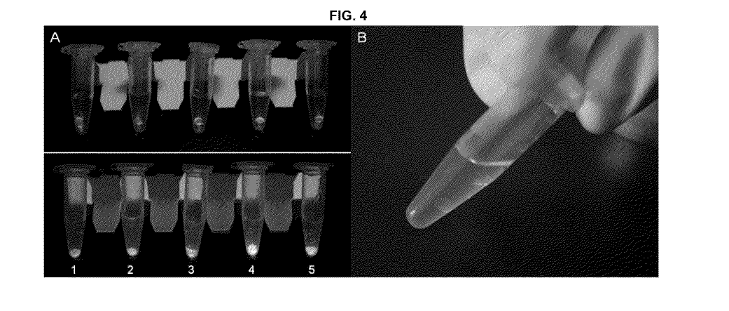

In addition, in the preparation of a nucleic acid gel matrix, components necessary for transcription and translation were included to prepare of a nucleic acid gel matrix. The nucleic acid gel matrix including the components necessary for transcription and translation was observed by staining a nucleic acid dye, such as SYBR green I. The results are shown in FIG. 4. From the results of FIG. 4, it was confirmed that five types of a nucleic acid gel matrix (control group) including no component in the first micro centrifuge tube, a nucleic acid gel matrix including a T7 RNA polymerase and a transcription buffer in the second micro centrifuge tube, a nucleic acid gel matrix including a T7 RNA polymerase, a transcription buffer and an NTP mixed solution in the third micro centrifuge tube, a nucleic acid gel matrix including only an NTP mixed solution in the fourth micro centrifuge tube, and a nucleic acid gel matrix including only a T7 RNA polymerase in the fifth micro centrifuge tube were well formed, and it was also easily confirmed with the naked eye that, as shown in FIG. 4(B), a nucleic acid gel matrix was formed.

### Example 2

### Nucleic Acid Gel Matrix for Synthesizing a Nucleus Mimetic Cell-Free Protein

2-1. Formation of Surface

To more effectively mimic a natural functional protein production capacity through nucleus mimicking, morphological mimicking of a cell nucleus had to be performed first. It was seen that, when an inside of a nucleus was understood to have a spherical or oval shape, a relative surface area was increased based on the same volume. Since a gene in the nucleic acid gel matrix having a large surface area had an increased probability to closely react with protein transcription-translation factors provided from an outside, more effective protein production was ultimately possible.

To prepare such a nucleic acid gel matrix, in the present invention, the characteristic of hydrophilic water minimizing surface tension on the superhydrophobic surface was used. Since a hydrophilic nucleic acid gel precursor was freely formed in a spherical shape on the superhydrophobic surface, simplicity of the preparing process was expected. Moreover, for comparison, the gel matrix was also prepared in the type of a rectangular gel pad, and thus a glass substrate simply providing a positive charge surface was used.

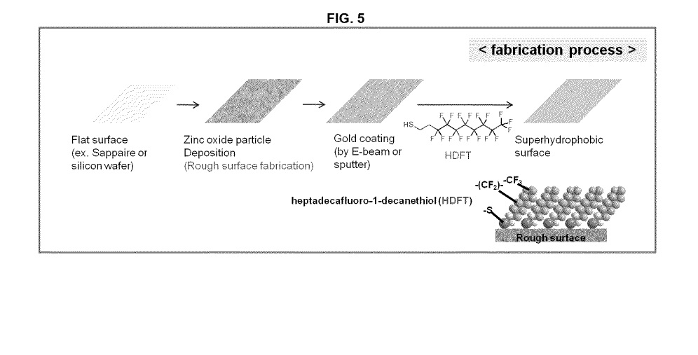

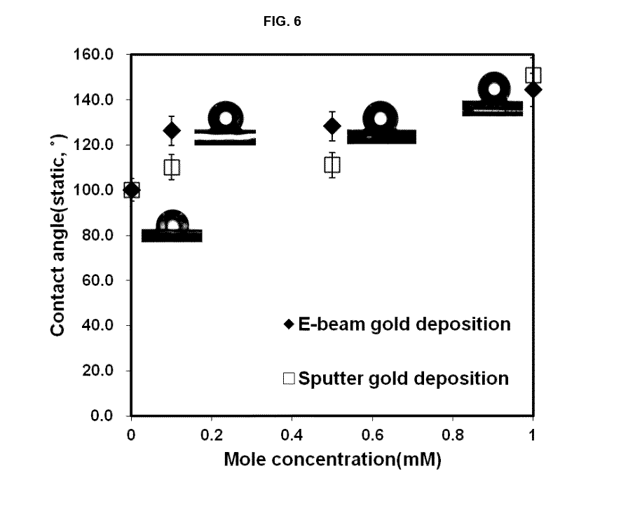

To prepare the superhydrophobic surface, a 5 nm gold thin film was formed by forming a rough surface by depositing zinc oxide (ZnO) particles on a sapphire substrate, and coating the surface with gold through e-beam lithography or sputtering. In addition, finally, various concentrations of HDFT solutions were treated at room temperature for 15 minutes, and the resulting surface was washed with dichloromethane for 2 minutes, thereby forming the superhydrophobic surface. A schematic diagram showing the preparing method is shown in FIG. 5. The hydrophobicity according to an HDFT concentration was measured using a contact angle. The results are shown in FIG. 6.

As shown in FIG. 6, it was confirmed that when HDFT was not treated, the contact angle was approximately 80 degrees, when 0.1 mM and 0.5 mM of HDFTs were treated, the contact angle was approximately 120 degrees, and when 1 mM of HDFT was treated, the contact angle was approximately 150 degrees. In addition, it was confirmed that, when 1 mM of HDFT was treated, the contact angle became larger by coating the surface with gold through vacuum deposition. According to the result, it was confirmed that the superhydrophobic surface can be formed by treating the surface with 1 mM of HDFT after the gold treatment through vacuum deposition.

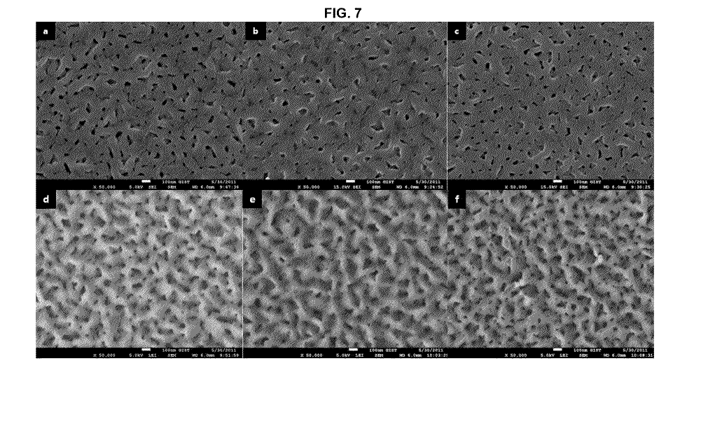

The superhydrophobic surface prepared through the treatment of 1 mM of HDFT was observed using a scanning electron microscope (SEM). The result is shown in FIG. 7.

As shown in FIG. 7, it was confirmed that rough surfaces were formed on the sapphire substrates both when the substrates were observed from above after zinc oxide particles were deposited (refer to FIG. 7(a)) and when the substrates were inclined at 45 degrees and observed (refer to FIG. 7(d)), and a gold membrane was covered on the rough surface when gold was deposited on the rough surface (refer to FIGS. 7(b) and (e)). In addition, the results of forming the superhydrophobic surfaces using HDFT were confirmed as shown in FIGS. 7(c) and (f).

2-2. Preparation of Nucleic Acid Gel Matrix

As shown in FIG. 3, a nucleic acid matrix was prepared by binding a recombinant plasmid including a targeting gene within an X-type nucleic acid nano structure based on an X-type nucleic acid nano structure.

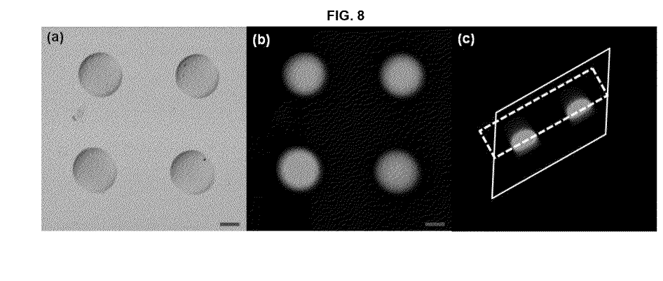

The X-type nucleic acid nano structure of Example 1-1 and the recombinant plasmid of Example 1-2 were put on the superhydrophobic surface of Example 2-1 using a T4 DNA ligase, a 100-μm circular diameter was covered with a polydimethylsiloxane (PDMS) template mold having a groove of 30 μm to induce gelation at room temperature for at least four hours, and the PDMS template was removed, thereby preparing a nucleic acid gel matrix. A nucleic acid in the prepared oval-shaped nucleic acid gel matrix was stained with a nucleic acid dye, that is, SYBR green I, and observed using a fluorescent microscope. The results are shown in FIG. 8.

As shown in FIG. 8, it was confirmed that an oval-shaped (diameter: 100 μm) nucleic acid gel matrix for synthesizing a nucleus mimetic cell-free protein was prepared in the form of a three-dimensional gel matrix.

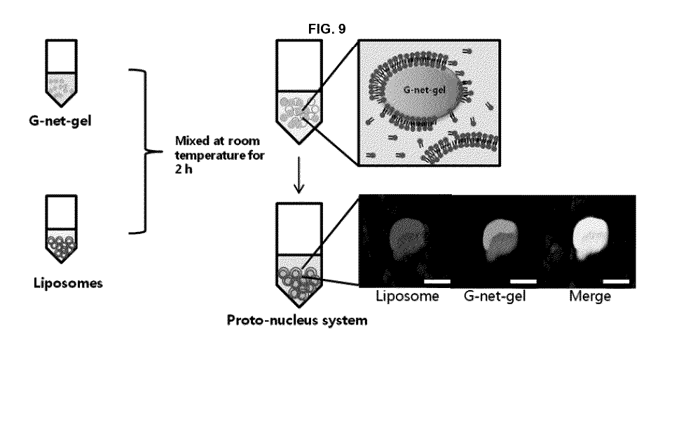

In addition, for nucleus mimicking, a structure for synthesizing a nucleus mimetic nucleic acid matrix-phase protein including lipid membrane components (DOPC, DOTAP, and Texas-red DHPE) as shown in FIG. 9 was prepared by a reaction with a liposome prepared with the lipid membrane components (DOPC, DOTAP, and Texas-red DHPE) at room temperature for 2 hours.

2-3. Prediction of Protein Production Efficiency of Nucleic Acid Gel Matrix for Synthesizing a Cell-Free Protein

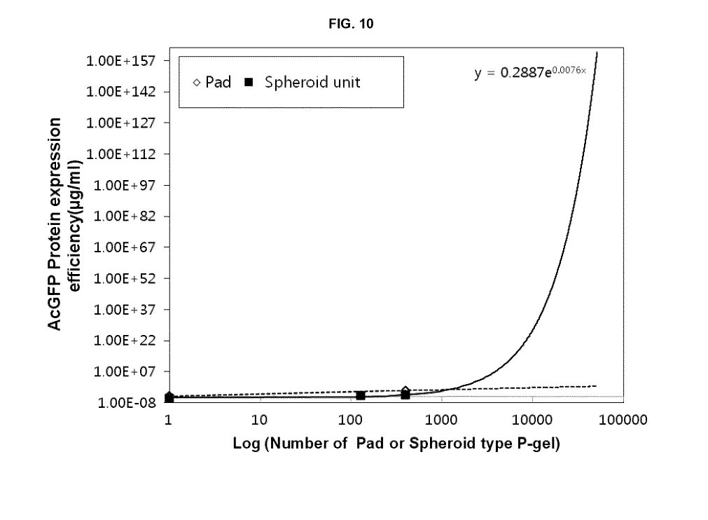

The oval-shaped nucleic acid gel matrix for synthesizing a cell-free protein was compared with a rectangular pad-type nucleic acid gel matrix in terms of protein production efficiency. On the assumption that one pad-type gel matrix has the same volume as 127 oval-shaped gel matrixes, an AcGFP protein expression level of the oval-shaped gel matrix was measured. From the equation y=0.2887e^{0.0076x} obtained from a measurement value, a protein expression level was predicted. The results are shown in FIG. 10.

As shown in FIG. 10, it was confirmed that 50,800 oval-shaped gel matrixes had effects increased approximately 20 times those of the 400 pad-type gel matrixes corresponding to the same volume. This means that as a surface area was increased due to the oval-shaped structure, factors such as internal DNAs were present on a surface of the gel matrix, a reaction efficiency with external protein production factors increased, and thus an amount of protein production could be considerably increased.

### Example 3

### Comparison with Solution-Phase Cell-Free Protein Synthesis System

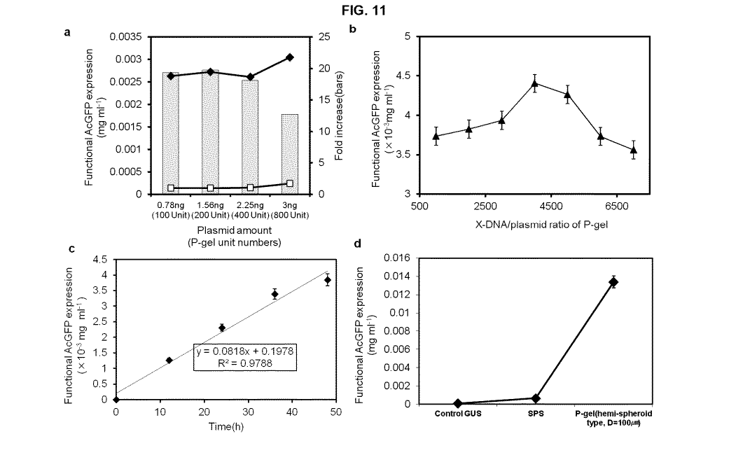

To compare an amount of synthesized protein of the system according to the present invention with that of a commercially available conventional solution-phase cell-free protein synthesis system, an AcGFP gene (an amount corresponding to 0.78, 1.56, 2.25, and 3 ng per unit. e.g., 0.78 ng of the AcGFP gene per 100 units)-added solution and a nucleic acid gel matrix were treated at 100, 200, 400, or 800 units, reacted at 24° C. for 24 hours, and then an amount of expressed proteins was measured using fluorescence. The results are shown in FIG. 11(a).

As shown in FIG. 11(a), when the solution-phase system (□) was treated with 800 units of the mixed solution, a protein expression level was approximately 0.23 μg/mL, but when the nucleic acid matrix-phase structure (♦) was treated with 100 units of the mixed solution, the protein expression level increased 20 times or more, and measured approximately 2.6 μg/mL. In addition, when the nucleic acid matrix-phase structure was treated with 800 units of the mixed solution, a protein expression level was 3.0 μg/mL, which represented that the protein expression level had considerably increased compared to the solution-phase system.

FIG. 11(b) shows comparison results of protein expression levels according to a ratio of an X-type nucleic acid nano structure to a recombinant plasmid included in a nucleic acid matrix-phase structure, which shows that when the ratio is 4,000:1, a protein expression level is the highest.

After the structure was prepared to have a ratio of the X-type nucleic acid nano structure to the recombinant plasmid of 4,000:1, 800 units of the mixed solution were treated thereto, and then a protein expression level in a continuous culture system was measured according to time. The results are shown in FIG. 11(c).

As shown in FIG. 11(c), it was identified that the expression level increased with time until 50 hours.

To identify the efficiency of the prepared structure for expressing a nucleic acid matrix-phase cell-free protein, using a β-glucuronidase (GUS) gene as a control group, a protein expression level was measured. The results are shown in FIG. 11(d).

As shown in FIG. 11(d), the nucleic acid matrix-phase structure showed a protein expression level 20 times that of the solution-phase system.

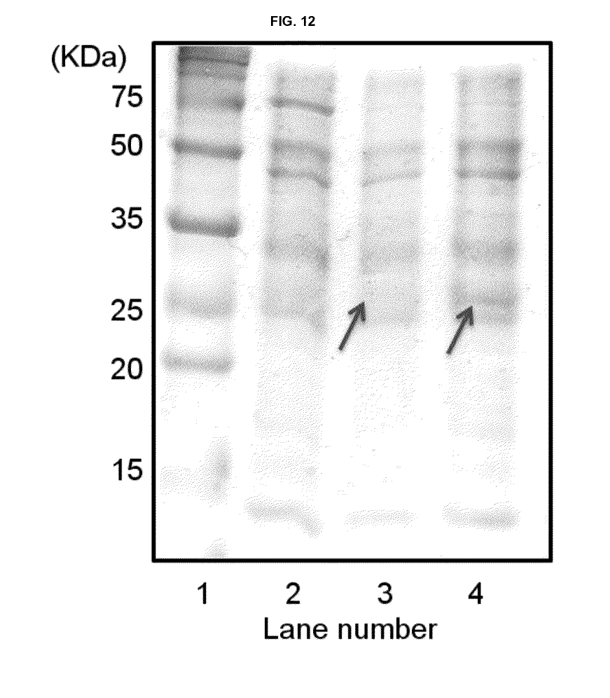

The expressed protein was indentified through SDS-PAGE. The results are shown in FIG. 12. As shown in FIG. 12, it was indentified that the GUS gene, which was the control group, was normally expressed (the second lane), and both the solution-phase system (the third lane) and the nucleic acid matrix-phase structure (the fourth lane) produced AcGFP (26 kDa).

Consequently, it was confirmed that a cell-free protein expression system having an expression level 20 times that of the conventional cell-free protein expression system can be developed using the nucleic acid matrix-phase structure, and when using the system, a high concentration protein can be stably formed for a long time.

A nucleic acid gel matrix for synthesizing a nucleus mimetic cell-free protein according to the present invention is similar to a natural genomic condensed structure in a cell formed by condensing DNA, and transcription and translation factors in a small place, and can produce a large amount of proteins. Therefore, when injected into a human body, the gel matrix can improve therapeutic effects such as stable anticancer treatment and treatment of inflammation In addition, the nucleus mimetic system prepared by the combination of the developed nucleic acid matrix and the lipid membrane components can be suggested as a next generation platform for future gene therapy since it is a naturally structured mimic which has hardly any toxicity or immune responses and therefore can replace an existing nuclear system of a cell.

While the invention has been shown and described with reference to certain exemplary embodiments thereof, it will be understood by those skilled in the art that various modifications in form and details may be made therein without departing from the spirit and scope of the invention as defined by the appended claims.

1. A nucleic acid gel matrix for synthesizing a nucleus mimetic cell-free protein, comprising:
an X-type nucleic acid nano structure, an expression plasmid including a DNA fragment, transcription and translation factors, and lipid membrane components.

2. The nucleic acid gel matrix of claim 1, wherein the X-type nucleic acid nano structure is formed by hybridizing DNA sequences of SEQ. ID. NOs: 1, 2, 3, and 4.

3. The nucleic acid gel matrix of claim 1, wherein the DNA fragment is an Aequorea coerulescens green fluorescent protein (AcGFP) gene.

4. The nucleic acid gel matrix of claim 1, wherein the DNA fragment is a gene related to cancer cell death or angiogenesis.

5. The nucleic acid gel matrix of claim 4, wherein the gene is selected from the group consisting of human sonic hedgehog (hShh), tumor necrosis factor-related apoptosis-inducing ligand (hTRAIL), human glail-derived neurotrophic factor (hGDNF), and mouse-stromal cell-derived factor-1α (SDF1α) genes.

6. The nucleic acid gel matrix of claim 1, wherein the DNA fragment is a gene related to production of a mussel adhesive protein (MAP).

7. The nucleic acid gel matrix of claim 6, wherein the gene is a fp-151 gene.

8. The nucleic acid gel matrix of claim 1, wherein the DNA fragment is a gene related to production of CD44, that is, a cancer stem cell marker.

9. The nucleic acid gel matrix of claim 1, wherein the expression vector including the DNA fragment is a plasmid in vitro expression (pIVEX) vector, that is, a wheat germ or E. coli based system.

10. The nucleic acid gel matrix of claim 1, wherein the transcription and translation factors include an RNA polymerase (T7 RNA polymerase), an ATP, an NTP mixed solution, a cell lysate, and an amino acid.

11. The nucleic acid gel matrix of claim 1, wherein the lipid membrane components are 1,2-dioleoyl-sn-glycero-3-phosphocholine (DOPC), 1,2-dioleoyl-3-trimethylammonium-propane (DOTAP), and 1,2-dihexadecanoyl-sn-glycero-3-phosphoethanolamine (Texas-red DHPE).

12. The nucleic acid gel matrix of claim 1, wherein the nucleic gel matrix has any of various shapes in which each size of width, length and height is 100 nm to 1,000 μm, or a maximum diameter is 100 μm or less.

13. A method of preparing a nucleic acid gel matrix for synthesizing a nucleus mimetic cell-free protein, comprising the step of:
(a) forming a superhydrophobic surface by depositing zinc oxide (ZnO) nanoparticles on a sapphire substrate, depositing gold on the surface through e-beam lithography or vacuum sputtering, and treating 1 mM of a heptadecafluoro-1-decanethiol (HDFT) solution at room temperature for 15 minutes;
(b) preparing an X-type nucleic acid nano structure by combining single strand nucleic acids of SEQ. ID. NO: 1, 2, 3, and 4 through complementary hybridization of base sequences;
(c) preparing a recombinant plasmid by combining a plasmid in vitro expression (pIVEX) vector with a DNA fragment using restriction enzymes and a T4 DNA ligase;
(d) preparing a nucleic acid gel precursor by combining the X-type nucleic acid nano structure, the recombinant plasmid, and the transcription and translation factors using a T4 DNA ligase;
(e) plating the nucleic acid gel precursor solution on the superhydrophobic surface, covering the surface with a PDMS template, and performing a reaction at room temperature for at least 4 hours; and
(f) reacting the prepared nucleic acid matrix with a liposome prepared with lipid membrane components (DOPC, DOTAP, and Texas-red DHPE) at room temperature for at least 2 hours.

14. The method of claim 13, wherein the DNA fragment is an AcGFP gene.

15. The method of claim 13, wherein the DNA fragment is a gene related to cancer cell death or angiogenesis.

16. The method of claim 15, wherein the gene is selected from the group consisting of human sonic hedgehog (hShh), tumor necrosis factor-related apoptosis-inducing ligand (hTRAIL), human glail-derived neurotrophic factor (hGDNF), and mouse-stromal cell-derived factor-1α (SDF1α) genes.

17. The method of claim 13, wherein the DNA fragment is a gene related to production of a mussel adhesive protein (MAP).

18. The method of claim 17, wherein the gene is a fp-151 gene.

19. The method of claim 13, wherein the DNA fragment is a gene related to production of CD44, that is, a cancer stem cell marker.

20. The method of claim 13, wherein the expression vector including the DNA fragment is a pIVEX vector, that is, a wheat germ or E. coli based system.

21. The method of claim 13, wherein the transcription and translation factors include an RNA polymerase (T7 RNA polymerase), an ATP, an NTP mixed solution, a cell lysate, and an amino acid.

22. The method of claim 13, wherein the lipid membrane components are 1,2-dioleoyl-sn-glycero-3-phosphocholine (DOPC), 1,2-dioleoyl-3-trimethylammonium-propane (DOTAP), and 1,2-dihexadecanoyl-sn-glycero-3-phosphoethanolamine (Texas-red DHPE).
